(19) United States
(12) Patent Application Publication
(10) Pub. No.: US 2024/0315910 A1
(43) Pub. Date: Sep. 26, 2024

(54) PERCEPTION SYSTEM FOR A LOWER BODY POWERED EXOSKELETON

(71) Applicant: Boston Dynamics, Inc., Waltham, MA (US)

(72) Inventors: Shervin Talebi, Wayland, MA (US); Ran Tao, Milpitas, CA (US); John Aaron Saunders, Arlington, MA (US); Dong Jin Hyun, Suwon-si (KR); San In Park, Brookline, MA (US)

(73) Assignee: Boston Dynamics, Inc., Waltham, MA (US)

(21) Appl. No.: 18/545,255

(22) Filed: Dec. 19, 2023

Related U.S. Application Data
(60) Provisional application No. 63/453,212, filed on Mar. 20, 2023.

(51) Int. Cl. A61H 3/00; A61H 1/02; A61H 3/02

(52) U.S. Cl. CPC A61H 3/00; A61H 1/0262; A61H 3/02; A61H 2003/001; A61H 2003/007; A61H 2201/1604; A61H 2201/1635; A61H 2201/164; A61H 2201/165; A61H 2201/5023; A61H 2201/5043; A61H 2201/5048; A61H 2201/5058; A61H 2201/5061; A61H 2201/5092

(57) ABSTRACT

Systems and methods for a perception system for a lower body powered exoskeleton device are provided. The perception system includes a camera configured to capture one or more images of terrain in proximity to the exoskeleton device, an at least one processor. The at least one processor is programmed to perform footstep planning for the exoskeleton device based, at least in part, on the captured one or more images of terrain, and issue an instruction to perform a first action based, at least in part, on the footstep planning.

### CROSS-REFERENCE TO RELATED APPLICATIONS

This application claims the benefit under 35 U.S.C. § 119(e) to U.S. Provisional Patent Application No. 63/453,212, filed on Mar. 20, 2023, and titled, “PERCEPTION SYSTEM FOR A LOWER BODY POWERED EXOSKELETON,” the entire contents of which is incorporated by reference herein.

### FIELD OF THE INVENTION

This disclosure relates to a perception system for a lower body powered exoskeleton.

### BACKGROUND

Patients with impaired lower body sensation (e.g., due to spinal cord injuries) may use a lower body powered exoskeleton device to assist with standing and walking. The exoskeleton device may include one or more joints with actuators that enable a user of the device to take steps. The actuators may be controlled based on gait parameters manually input to a computer system in communication with the exoskeleton device. One or more helpers may assist the user of the exoskeleton device to facilitate balance and prevent falls while the user is using the device.

### SUMMARY

Lower body powered exoskeleton devices are designed to allow paraplegic patients to stand and walk. The usability of such devices may pose challenges, for example, due to gait parameters (e.g., step length, step height, step timing, etc.) typically being required to be adjusted manually. Additionally, the user of a lower body powered exoskeleton device (also referred to herein a “rider”) does not typically receive feedback on foot placement or balance from the device during use. As such, it is impractical for the rider to use the exoskeleton device without assistance from one or more helpers to support their movements as they walk. Some embodiments of the present disclosure relate to a perception system for a lower body powered exoskeleton system. The perception system may be configured to detect aspects of the terrain in front of the rider and provide automatic gait adjustments based on the terrain. In some embodiments, the perception system may be further configured to provide feedback to the rider. For instance, the feedback may include information about an operation of the exoskeleton device (e.g., footstep and/or crutch target locations) and/or balance information associated with the device and/or rider.

In some embodiments, the invention features a perception system for a lower body powered exoskeleton device. The perception system includes a camera configured to capture one or more images of terrain in proximity to the exoskeleton device, and at least one processor. The at least one processor is programmed to perform footstep planning for the exoskeleton device based, at least in part, on the captured one or more images of terrain, and issue an instruction to perform a first action based, at least in part, on the footstep planning.

In one aspect, issuing an instruction to perform a first action includes issuing an instruction to provide feedback to a user of the exoskeleton device based, at least in part on the footstep planning. In another aspect, issuing an instruction includes sending information to a visualization system configured to provide the feedback to the user. In another aspect, the visualization system includes an augmented reality (AR) system. In another aspect, performing footstep planning for the exoskeleton device includes determining a location of one or more footstep targets, and sending information to the visualization system includes sending information associated with the location of the one or more footstep targets. In another aspect, performing footstep planning for the exoskeleton device includes determining a location of one or more crutch targets to place a crutch configured to be used with the exoskeleton device, and sending information to the visualization system includes sending information associated with the location of the one or more crutch targets.

In another aspect, issuing an instruction to provide feedback includes issuing an instruction to an audio system to output audio including the feedback. In another aspect, issuing an instruction to provide feedback includes issuing an instruction to a haptic system to output haptic feedback to the user. In another aspect, issuing an instruction to provide feedback includes issuing an instruction to provide at least two of visual, audio, and haptic feedback to the user. In another aspect, issuing an instruction to perform a first action includes issuing an instruction to a controller of the exoskeleton device to set one or more gait parameters of the exoskeleton device based, at least in part on the footstep planning. In another aspect, the one or more gait parameters of the exoskeleton are selected from the group consisting of step length, step height, and step timing.

In another aspect, performing footstep planning includes determining at least one footstep target location for the exoskeleton device in which a foot of exoskeleton device has full contact with a surface of the terrain. In another aspect, the terrain includes a step, and performing footstep planning includes determining at least one footstep target location for the exoskeleton device on the step.

In another aspect, the at least one processor is further programmed to perform balance estimation associated with the exoskeleton device and issue an instruction to perform a second action based, at least in part, on the balance estimation. In another aspect, issuing an instruction to perform the second action includes issuing an instruction to provide feedback to a user of the exoskeleton device based, at least in part, on the balance estimation. In another aspect, issuing an instruction to provide feedback includes sending information to a visualization system configured to display the feedback to the user. In another aspect, the visualization system includes an augmented reality (AR) system. In another aspect, performing balance estimation associated with the exoskeleton device includes determining a balance state associated with the exoskeleton device, and sending information to the visualization system includes sending information associated with the balance state. In another aspect, determining a balance state associated with the exoskeleton device includes determining a numerical value for the balance state associated with the exoskeleton device, and the information associated with the balance state includes information associated with the numerical value. In another aspect, the information associated with the numerical value includes a balance meter that indicates to the user whether the balance state is sufficient to perform a next leg swing of the exoskeleton device. In another aspect, information associated with the balance state further includes information of how to improve the balance state when it is determined that the balance state is not sufficient to perform a next leg swing of the exoskeleton device. In another aspect, determining a balance state of the exoskeleton device is performed, based at least in part, on force information received from at least one sensor. In another aspect, the at least one sensor includes a foot force sensor located on a foot portion of the exoskeleton device, and the force information includes force information received from the foot force sensor. In another aspect, the at least one sensor includes a force sensor arranged on a crutch configured to be used with the exoskeleton device, and the force information includes force information received from the force sensor arranged on the crutch.

In another aspect, issuing an instruction to provide feedback includes issuing an instruction to an audio system to output audio including the feedback. In another aspect, issuing an instruction to provide feedback includes issuing an instruction to an haptic system to output haptic feedback to the user. In another aspect, issuing an instruction to provide feedback includes issuing an instruction to provide at least two of visual, audio, and haptic feedback to the user. In another aspect, the perception system further includes an inertial measurement unit (IMU), and issuing an instruction to provide feedback to the user of the exoskeleton device includes issuing an instruction to provide feedback based, at least in part on an output of the IMU.

In some embodiments, the invention features a method of providing assistive feedback to a user of a lower body powered exoskeleton device. The method includes receiving one or more images of terrain in front of the exoskeleton device, performing, by at least one processor, footstep planning for the exoskeleton device, the footstep planning being performed based, at least in part, on the one or more images of terrain, and providing assistive feedback to the user of the exoskeleton device based, at least in part, on the footstep planning.

In one aspect, providing assistive feedback to the user includes sending information to a visualization system configured to provide the assistive feedback to the user. In another aspect, the visualization system includes an augmented reality (AR) system. In another aspect, performing footstep planning for the exoskeleton device includes determining a location of one or more footstep targets, and providing assistive feedback to the user includes displaying the location of the one or more footstep targets. In another aspect, performing footstep planning for the exoskeleton device includes determining a location of one or more crutch targets to place a crutch configured to be used with the exoskeleton device, and providing assistive feedback to the user includes displaying the location of the one or more crutch targets.

In another aspect, providing assistive feedback to the user includes outputting audio including the assistive feedback. In another aspect, providing assistive feedback to the user includes outputting haptic feedback to the user. In another aspect, providing assistive feedback to the user includes providing at least two of visual, audio, and haptic feedback to the user. In another aspect, providing assistive feedback to the user includes setting one or more gait parameters of the exoskeleton device based, at least in part on the footstep planning. In another aspect, the one or more gait parameters of the exoskeleton are selected from the group consisting of step length, step height, and step timing.

In another aspect, performing footstep planning includes determining at least one footstep target location for the exoskeleton device in which a foot of exoskeleton device has full contact with a surface of the terrain. In another aspect, the terrain includes a step, and performing footstep planning includes determining at least one footstep target location for the exoskeleton device on the step.

In another aspect, the method further includes performing balance estimation associated with the exoskeleton device, and providing assistive feedback to the user of the exoskeleton device is further based, at least in part, on the balance estimation. In another aspect, providing assistive feedback to the user includes sending information to a visualization system configured to provide the assistive feedback to the user. In another aspect, the visualization system includes an augmented reality (AR) system. In another aspect, performing balance estimation associated with the exoskeleton device includes determining a balance state associated with the exoskeleton device, and providing assistive feedback to the user includes providing an indication of the balance state. In another aspect, determining a balance state associated with the exoskeleton device includes determining a numerical value for the balance state associated with the exoskeleton device, and providing an indication of the balance state includes providing an indication of the numerical value. In another aspect, the indication of the numerical value includes a balance meter that indicates to the user whether the balance state is sufficient to perform a next leg swing of the exoskeleton device. In another aspect, providing an indication of the balance state further includes providing an indication of how to improve the balance state when it is determined that the balance state is not sufficient to perform a next leg swing of the exoskeleton device. In another aspect, determining a balance state of the exoskeleton device is performed, based at least in part, on force information received from at least one sensor. In another aspect, the at least one sensor includes a foot force sensor located on a foot portion of the exoskeleton device, and the force information includes force information received from the foot force sensor. In another aspect, the at least one sensor includes a force sensor arranged on a crutch configured to be used with the exoskeleton device, and the force information includes force information received from the force sensor arranged on the crutch.

In another aspect, providing assistive feedback to the user includes outputting audio including the assistive feedback. In another aspect, providing assistive feedback to the user includes outputting haptic feedback to the user. In another aspect, providing assistive feedback to the user includes providing at least two of visual, audio, and haptic feedback to the user. In another aspect, providing assistive feedback to the user of the exoskeleton device includes providing assistive feedback based, at least in part on an output of an inertial measurement unit (IMU).

In some embodiments, the invention features a system. The system includes a lower body powered exoskeleton device configured to be worn by a user, a perception system configured to capture one or more images of terrain in proximity to the exoskeleton device, an augmented reality (AR) system configured to be worn by the user, and at least one processor. The at least one processor is programmed to perform footstep planning for the exoskeleton device based, at least in part, on the one or more images of terrain and send first information based on a result of the footstep planning to the AR system for presentation by the AR system to the user.

In one aspect, the system further includes a foot force sensor configured to sense foot force information, and the at least one processor is further programmed to perform balance estimation based, at least in part, on the foot force information. In another aspect, the at least one processor is further programmed to send second information based on a result of the balance estimation to the AR system for presentation by the AR system to the user. In another aspect, performing balance estimation associated with the exoskeleton device includes determining a balance state associated with the exoskeleton device, and the second information includes information associated with the balance state. In another aspect, determining a balance state associated with the exoskeleton device includes determining a numerical value for the balance state associated with the exoskeleton device, and the second information includes information associated with the numerical value. In another aspect, the AR system is configured to display a balance meter to the user, the balance meter indicating, based at least in part, on the second information, whether the balance state is sufficient to perform a next leg swing of the exoskeleton device. In another aspect, the AR system is further configured to indicate an instruction to the user how to improve the balance state when it is determined that the balance state is not sufficient to perform a next leg swing of the exoskeleton device.

In another aspect, the perception system is mounted to a pelvis of the exoskeleton device. In another aspect, performing footstep planning for the exoskeleton device includes determining a location of one or more footstep targets, and sending information to the AR system includes sending information associated with the location of the one or more footstep targets. In another aspect, performing footstep planning for the exoskeleton device includes determining a location of one or more crutch targets to place a crutch configured to be used with the exoskeleton device, and sending information to the AR system includes sending information associated with the location of the one or more crutch targets.

In another aspect, the at least one processor is further programmed to set one or more gait parameters of the exoskeleton device based, at least in part on the footstep planning. In another aspect, the one or more gait parameters of the exoskeleton are selected from the group consisting of step length, step height, and step timing. In another aspect, performing footstep planning includes determining at least one footstep target location for the exoskeleton device in which a foot of exoskeleton device has full contact with a surface of the terrain. In another aspect, the terrain includes a step, and performing footstep planning includes determining at least one footstep target location for the exoskeleton device on the step. In another aspect, the system further includes an inertial measurement unit (IMU), and the at least one processor is further programmed to send second information based on output of the IMU to the AR system.

### BRIEF DESCRIPTION OF DRAWINGS

The advantages of the invention, together with further advantages, may be better understood by referring to the following description taken in conjunction with the accompanying drawings. The drawings are not necessarily to scale, and emphasis is instead generally placed upon illustrating the principles of the invention.

FIG. 1 illustrates components of a lower body powered exoskeleton device that may be used according to an illustrative embodiment of the invention.

FIG. 2 illustrates components of a perception system that may be used with a lower body powered exoskeleton device, according to an illustrative embodiment of the invention.

FIG. 3 is a block diagram of components of a perception system that may be used with a lower body powered exoskeleton device, according to an illustrative embodiment of the invention.

FIG. 4 shows an example visualization of a footstep planning modeling process for a perception-informed exoskeleton device, according to an illustrative embodiment of the invention.

FIGS. 5A and 5B show visualizations of feedback information displayed using an augmented reality system, according to an illustrative embodiment of the invention.

FIG. 6 illustrates how a user of a perception-informed exoskeleton device may visualize feedback information during use of the exoskeleton device, according to an illustrative embodiment of the invention.

FIG. 7 is a flowchart of a process for using a perception system to perform footstep planning and/or balance estimation for a lower body powered exoskeleton device, according to an illustrative embodiment of the invention.

FIG. 8 is a flowchart of a process for generating visual feedback associated with footstep planning and/or balance estimation, according to an illustrative embodiment of the invention.

FIG. 9 illustrates an example configuration of a robotic device, according to an illustrative embodiment of the invention.

### DETAILED DESCRIPTION

A lower body powered exoskeleton device (also referred to herein simply as an “exoskeleton device”) is a robotic device that enables patients with lower limb impairments to stand and walk. As described above, some conventional exoskeleton devices present safety and/or usability challenges to the extent that the devices typically cannot be used without the support of one or more helpers. Additionally, the gait parameters of the exoskeleton device are typically configured manually, making usability cumbersome and slow. Some embodiments of the present disclosure are directed to systems and techniques for improving the usability of an exoskeleton device by providing feedback to the system and/or the user of the system based on perception of one or more aspects of the user's environment.

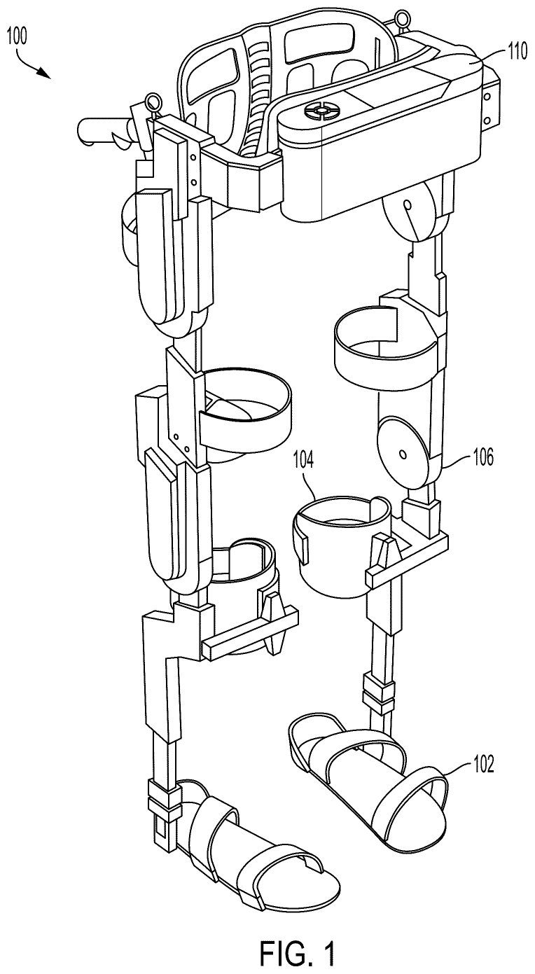

FIG. 1 illustrates an example of an exoskeleton device 100 that may be used in accordance with some embodiments of the present disclosure. Exoskeleton device 100 may include a foot portion 102 within which a user may place their feet and one or more leg portions 104 within which a user may place their legs. Exoskeleton device 100 may also include one or more joints that enable the exoskeleton legs to articulate about the joints. Each of the joints may be associated with an actuator 106 configured to be controlled by a hardware controller 110 to provide a desired amount of articulation of the joint to enable the exoskeleton device 100 to take a step. As described above, the hardware controller 110 of some conventional exoskeleton devices may receive as input gait parameters from an external computing device (e.g., a laptop computer or other computing device). A helper assisting a user of the exoskeleton device 100 may manually input one or more gait parameters (e.g., step length, step height, step timing, etc.) into the external computing device. Information based on the input gait parameters may be provided from the external computing device to hardware controller 110 arranged on exoskeleton device 100. Upon receiving the gait parameter information, the hardware controller 110 may be configured to send control commands to the actuator(s) 106 to execute a step operation. When the user of the exoskeleton device 100 is not walking on a flat surface, each step operation of the device may need to be planned and appropriate gait parameters input by a helper to ensure that the next step, when taken, results in a stable placement of the exoskeleton and its rider in the environment.

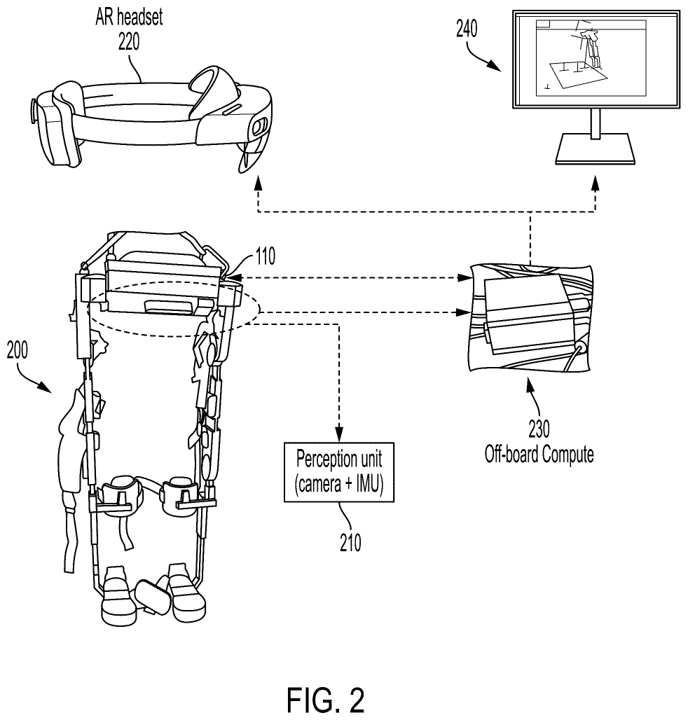

The inventors have recognized and appreciated that widespread adoption and/or overall usability of exoskeleton devices may be hindered by their non-autonomous operation and lack of feedback provided to the user about their operation. FIG. 2 illustrates an example of an exoskeleton device 200 associated with a perception system 210 configured to determine information about the environment of the exoskeleton device, in accordance with some embodiments of the present disclosure. For instance, perception system 210 may include one or more cameras and an inertial measurement unit (IMU). Perception system 210 may be configured to capture one or more images of terrain in proximity to exoskeleton device 200. The one or more images captured by the perception system 210 may be provided to compute device 230 that includes one or more processors configured to process the image(s). For instance, compute device 230 may be configured to determine terrain information associated with the environment of the exoskeleton device 200 to enable footstep planning and/or balance estimation, as described in more detail below. Although compute device 230 is shown in FIG. 2 as being located external to exoskeleton device 200, in some embodiments, compute device 230 may be implemented onboard exoskeleton device 200 and/or be located at, least in part, on another computing device in communication with exoskeleton device 200. For example, in some embodiments, compute device 230 may be implemented, at least in part, on visualization device 220, described in more detail below, or on another wearable computing device (not shown) worn by the user of the exoskeleton device 220.

Based, at least in part, on an output of processing the image(s), compute device 230 may be configured to initiate one or more actions. For instance, compute device 230 may send information based on the processed images to controller 110 onboard the exoskeleton device 200 to control operation of one or more actuators of the exoskeleton device, as described above. For instance, when the compute device 230 determines that the terrain in front of the exoskeleton device 200 includes a step, compute device 230 may be configured to provide information (e.g., control instructions) to controller 110, which may in turn automatically adjust one or more gait parameters of the exoskeleton device 200 to enable the exoskeleton device to step onto the step. More generally, feedback provided by compute device 230 may be provided to controller 110 to facilitate navigation of exoskeleton device 200 across terrain in the environment of the exoskeleton device by automatically selecting gait parameter(s) appropriate for the detected terrain. In some embodiments, compute device 230 may be configured to update gait parameters used by a controller of exoskeleton device 200, but control of the exoskeleton device (e.g., to initiate taking an next step) may be left up to the user to control. For instance, as described in more detail below, a user may be provided with feedback that enables them to determine when it is safe to take a next step.

In some embodiments, compute device 230 may be configured to provide feedback to the user of the exoskeleton device 200 to facilitate the user's understanding of how the exoskeleton device 200 may be controlled. For instance, visual feedback determined based, at least in part, on the processed images may be provided via a visualization device 220 communicatively coupled to the compute device 230. In some embodiments, visualization device 220 may be implemented as an augmented reality (AR) or mixed reality (MR) device configured to be worn by the user of the exoskeleton device 230. In some embodiments, visualization device 220 may be implemented as a display on a computing device (e.g., a display of a smartphone). When implemented as an AR device, visualization device 220 may be configured to display visual feedback, examples of which include, planned footstep locations and/or crutch placements, overlaid on a view of the environment of the exoskeleton device 200. For instance, the AR device may be implemented as see through glasses that project visualizations onto the scene observed by the user. Additionally or alternatively other visual feedback (e.g., balance information) may be provided from compute device 230 to visualization device 220 for display to the user.

In some embodiments, compute device 230 may be configured to perform footstep planning based, at least in part, on processing the images captured by perception system 210. The footstep planning may involve generating one or more models of the environment of the exoskeleton device 200. For instance, the compute device 230 may be configured to generate a terrain map of the environment and the footstep planning process may determine one or more candidate locations within the terrain map for the exoskeleton device 200 to take a next step and/or for a user to place a crutch. As shown in FIG. 2, a representation of the footstep planning process may be output to a display 240 for visualization of the modeling process. Visualization of the modeling process may be useful, for example, to enable a developer to understand how the compute device 230 is interpreting various aspects of the terrain relative to the exoskeleton device to be used in footstep planning and/or balance estimation, as described herein.

In some embodiments, compute device 230 may be configured to provide feedback in addition to or alternatively to visual feedback. For instance, compute device 230 may be configured to provide audio feedback and/or haptic feedback that informs the user about one or more operations of the exoskeleton device 200. For example, compute device 230 may be configured to provide audio feedback to one or more speakers to inform the user that the exoskeleton device is ready to take a next step, that the user should lean to the right or the left to improve balance of the exoskeleton device, or to provide any other suitable audio output. Similarly, compute device 230 may be configured to communicate with one or more haptic devices (e.g., a vibratory device located on a crutch used by the user of exoskeleton device 200) to provide haptic feedback regarding an operation of the exoskeleton device 200. In some embodiments, compute device 230 is configured to provide feedback to the user using at least two modalities (e.g., at least two of audio, visual, and haptic feedback).

In some embodiments, one or more of exoskeleton device 200, perception system 210, visualization device 220 or another device (e.g., a headset) used in combination with one of those devices, may include one or more microphones configured to capture audio data (e.g., speech information such as speech commands) provided by a user of exoskeleton device 200. The received audio data may be provided to compute device 230 for processing for use in performing one or more actions (e.g., sending a control instruction to a controller of the exoskeleton device 200 to take a next step).

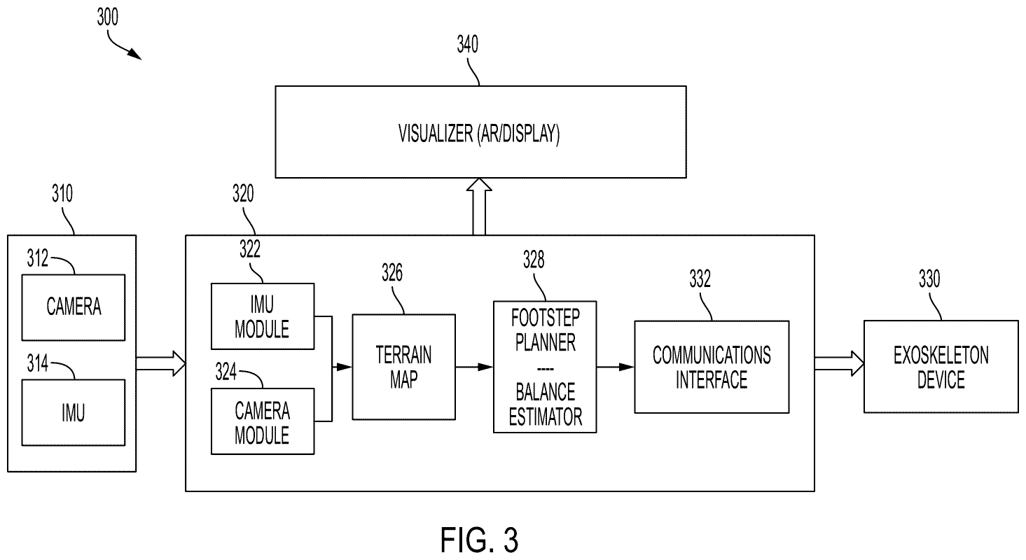

FIG. 3 illustrates a system 300 in accordance with an illustrative embodiment of the present disclosure. System 300 includes perception components 310 including one or more cameras 312 and an inertial measurement unit (IMU) 314. In some embodiments, perception components 310 may be implemented using perception system 210 described in connection with FIG. 2. In some embodiments, perception components 310 may be mounted on the pelvis of the exoskeleton device 330 to enable capture of images of the terrain in front of the exoskeleton device. In some embodiments, the IMU 314 may be used to determine the orientation of camera 312 relative to the ground.

System 300 may also include processing components 320. Processing components 320 may receive input from perception components 310 and provide output to exoskeleton device 330 and/or visualizer 340, as described further below. In some embodiments, processing components 320 may be implemented on compute device 230 described in connection with the system of FIG. 2. Processing components 320 includes IMU module 322 configured to process information received from IMU 314. Processing components 320 also includes camera module 324 configured to process information (e.g., images) received from camera(s) 312. As shown in FIG. 3, the output of IMU module 322 and camera module 324 may be used to generate a terrain map 326 of the terrain sensed by perception components 310. For instance, the terrain map may include height information associated with detected objects in the captured images.

Terrain map 326 may be provided as input to a footstep planning and/or balance estimation process 328. As described above, to enable a user to walk with a lower body powered exoskeleton device, gait parameters (e.g., step length, step height, step timing, etc.) for the exoskeleton device are typically entered manually by a helper. In some embodiments, one or more (e.g., all) gait parameters for the exoskeleton device 330 may be determined automatically (e.g., without user input) based, at least in part, on terrain map 326. Some embodiments implement a footstep planning process that determines footstep target locations for a next step of the exoskeleton device based on the terrain map 326, and corresponding gait parameters may be determined based, at least in part, on the planned footstep target locations.

The footstep target locations may be determined based on information about the exoskeleton device (e.g., the size of the feet of the exoskeleton device, the allowable step size range of the exoskeleton device), and information about the terrain. The inventors have recognized that stability of the exoskeleton device on the terrain may be improved by ensuring that most or all of the foot of the exoskeleton device is in contact with the terrain. For instance, when the terrain includes a step, it may be more stable for the exoskeleton device to place most or all of its foot on the step rather than having a portion of the foot hanging off the edge of the step. Accordingly, in some embodiments, a footstep target location for the exoskeleton device may be determined such that the foot of the exoskeleton has full contact with the surface of the terrain as specified in terrain map 326. Determining whether a foot of the exoskeleton device has full contact with the surface of the terrain may be performed in any suitable manner. In some embodiments, each of a plurality of points on the foot of the exoskeleton device may be modeled and the distance between those points and the terrain represented in terrain map 326 may be minimized. If it is determined that a candidate footstep target location does not place the foot in a location on the terrain with full contact, a different candidate footstep target location that does have full contact with the surface of the terrain may be selected. For instance, the candidate footstep location may be moved forward, backward or in another direction until a location in which the foot has full contact with the surface is found.

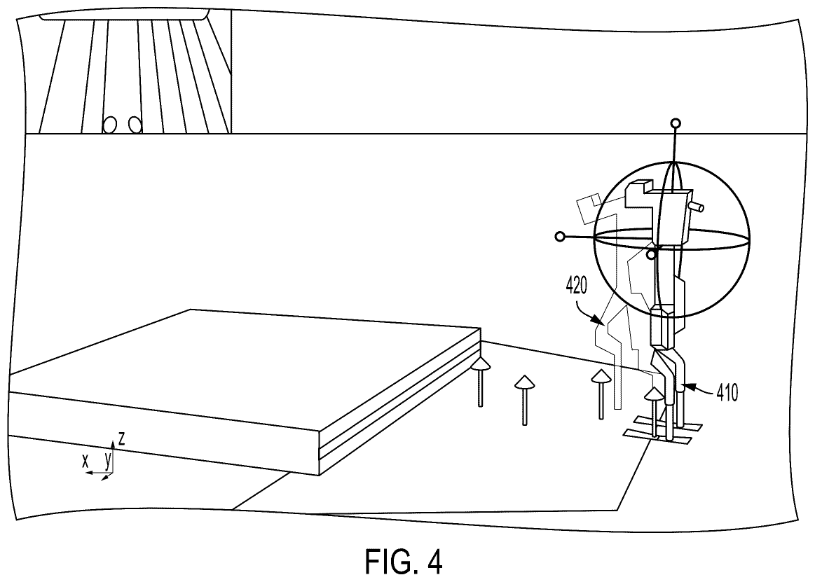

FIG. 4 illustrates a visualization of an output of a footstep planning process in accordance with some embodiments. As shown, a current location 410 of the exoskeleton device and a future location 420 of the exoskeleton device may be modeled based on one or more planned movements of the exoskeleton device and/or the crutches that the user of the exoskeleton device may use to improve stability. A terrain map generated based on images captured from the perception system may be used to identify footstep target locations and/or crutch target locations, as output of the footstep planning process. A visualization of the footstep planning process, such as that shown in FIG. 4, may be useful as a development tool to better understand how the modeling process is working based on various assumptions and conditions being provided as input to the model.

After performing footstep planning in process 328, one or more gait parameters output from the footstep planning process may be provided to a controller of exoskeleton device 330. For instance, information about the step size, step height, and/or step timing may be provided to exoskeleton device 330 via communications interface 332. In some embodiments, communications interface 332 is implemented as a wireless communications interface (e.g., a WiFi interface) between processing components 320 and a controller of exoskeleton device 330. When processing components 320 are implemented onboard exoskeleton device 330 communications interface 332 may be implemented as a wired communications interface between processing components 320 and a controller of exoskeleton device 330. In this way, one or more gait parameters may be determined and updated, at least in part, on perceived information about the terrain in which the exoskeleton device is operating rather than relying on a helper to manually determine and input the values. Such an automated approach may have several advantages compared to a manual entry approach. For example, when inputting step size, a helper typically must select from a discrete set of step sizes (e.g., 10 cm, 15 cm, 18 cm, 22 cm) despite the exoskeleton device being capable of taking steps at any step size within an allowable range (e.g., 1-35 cm). By requiring the helper to select from among a discrete set of step sizes the footstep target locations are considerably limited. By contrast, when the step size is automatically determined based, at least in part, on terrain map 326, any step size within the allowable step size range may be selected subject to certain constraints (e.g., full foot contact with the surface of the terrain). Additionally, an automatic updating of gait parameters using the techniques described herein may enable footstep planning that incorporates a level of dynamics that is not possible when manual input of gait parameters is required. For instance, an automatic update of gait parameters based on a perception of the terrain may enable the user to step faster and/or with more balance compared to the conventional manual input approach.

In some embodiments, the footstep planning process may be informed, at least in part, on a balance estimation of the exoskeleton device. For instance, in planning a next step for the left foot, a balance estimation associated with a left leg swing to accomplish the next left foot placement may be used to determine a footstep target location for the left foot. The balance estimation may be determined based, at least in part, on static forces applied to the parts of the exoskeleton device in contact with the ground (e.g., the exoskeleton device feet) and/or one or more crutches used to provide stability to the user of the exoskeleton device. In some embodiments, the balance estimation may be determined based, at least in part, on a sequence of movements with the exoskeleton device feet and/or crutches (e.g., left foot->left crutch->right foot->right crutch, or some other sequence). In some embodiments, exoskeleton device 330 includes at least one foot sensor arranged on a foot of the device. Using information from the foot sensor(s) and information from the perception components 310, the center of mass of the entire system (e.g., the exoskeleton device and the user) may be determined. Information from the perception components 310 may be used to localize a support polygon in the environment, and it may be determined whether the center of mass of the entire system is within the localized support polygon. When it is determined that the center of mass is within the support polygon, it may be determined that the current balance state is good and it is safe for the user to swing the leg of the exoskeleton to execute a next step. Information regarding the current balance state may be provided to the user, as described in more detail below.

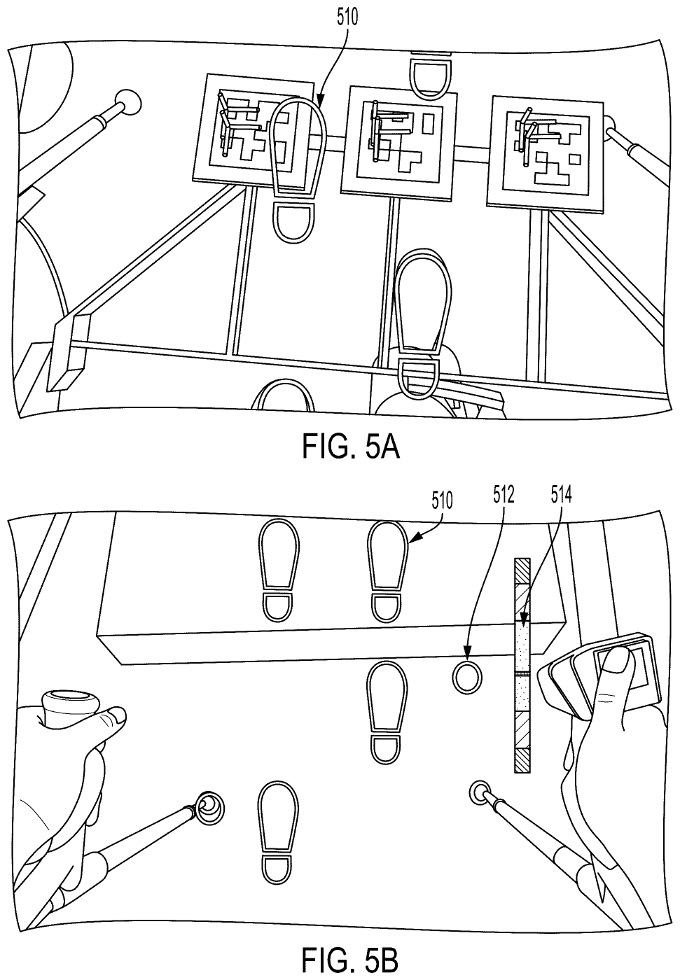

In some embodiments, information determined by footstep planning and/or balance estimation process 328 may be provided as feedback to the user of exoskeleton device 330 to enable the user to better understand the planned movements of the exoskeleton device. For instance, the feedback may be provided to visualizer 340, which in some embodiments, may be implemented as an augmented reality (AR) or mixed reality (MR) device that includes a display. The output of footstep planning and/or balance estimation process 328 may include one or more footstep target locations and/or one or more crutch target locations. This information may be provided to visualizer 340, which may be configured as a representation of the footstep target locations and/or the crutch target locations projected onto a scene of the environment as observed through the visualizer 340. FIGS. 5A and 5B illustrate examples of footstep target locations 510 and crutch target locations 512 projected as a visual overlay onto the environment directly in front of an exoskeleton device, in accordance with some embodiments. Providing visual feedback regarding footstep and/or crutch target information to the user of the exoskeleton device may enable the user to better understand and plan for upcoming movements of the device. Additionally, the user may be able to try and “land” the crutches in the visualized crutch target locations 512 during use of the device, possibly resulting in better stability as the exoskeleton device is used. In the example visualizations shown in FIGS. 5A and 5B, the footstep target locations and the crutch target locations are projected onto the environment in a manner that may require the user of the exoskeleton device to be looking down. The inventors have recognized that continuously looking down while using the exoskeleton device may be tiresome for the user. Accordingly, in some embodiments, the feedback information regarding footstep target and/or crutch target placement may be provided in a heads-up display that does not require the user to continuously look down.

In some embodiments, a sequence and amount of leg and crutch placement to improve or maximize stability (e.g., balance) may be determined. The inventors have recognized and appreciated that paraplegics that may use the exoskeleton device to stand and walk do not typically have a good sense of their balance. For example, they may not know what will happen regarding their balance when they swing their leg in the exoskeleton device. Indeed, the only information a user may receive about balance is how much pressure they exert on the crutches. For instance, they may perceive that more pressure exerted on the crutch corresponds to their center of mass being more forward (e.g., close to the crutch edge) and in such a situation they may be sufficiently balanced to perform a leg swing.

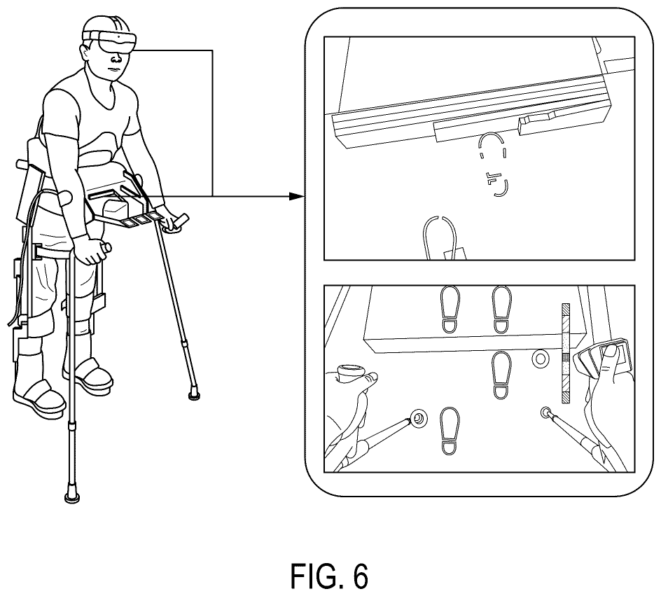

In some embodiments, an indication of the balance estimation of the user and exoskeleton device may be provided as visual feedback by visualizer 340. For instance, as shown in FIG. 5B, a balance meter 514 represents the current balance state of the rider and exoskeleton device. The output of a balance estimation process, as described above, may be a numerical value, and the balance meter 514 may translate the numerical value into a visualization that is easy for the user to understand their current balance state. For instance, the balance meter 514 may use different colors to represent how well the user riding in the exoskeleton device is currently balanced. For instance, the balance meter 514 may indicate a green range in which balance is good and a next step (e.g., a next leg swing) can be performed, and a red range in which balance is poor and a next step should not be attempted until a better balance is achieved. In some embodiments, after determining that the current balance state is sufficient to take a next step, the user of the exoskeleton device may press a button on one of their crutches to initiate taking the next step. In this way, the user of the exoskeleton device may experience more independence and control over operation of the exoskeleton device, which may improve its usability and/or adoption. In some embodiments, balance information feedback may also be provided in modalities other than providing visual feedback. For example, balance information feedback may be provided using auditory and/or haptic feedback to indicate a current balance state of the exoskeleton device. In some embodiments, the balance information feedback may provide the user with instructions on how to improve the balance state of the exoskeleton device when it is determined to be in a poor balance state (e.g., the center of mass of the rider and exoskeleton device is outside of a calculated support polygon). For instance, the balance information feedback may indicate to the user to shift their weight to the left or to the right to improve the balance state. The balance information feedback presented to the user may be updated due to changes in the balance state, and the user can continue to adjust their balance state until a good balance state has been achieved to be able to take a next step. FIG. 6 describes how a user of the exoskeleton device 330 wearing visualizer 340 may visualize footstep target location feedback and balance information feedback, in accordance with some embodiments of the present disclosure.

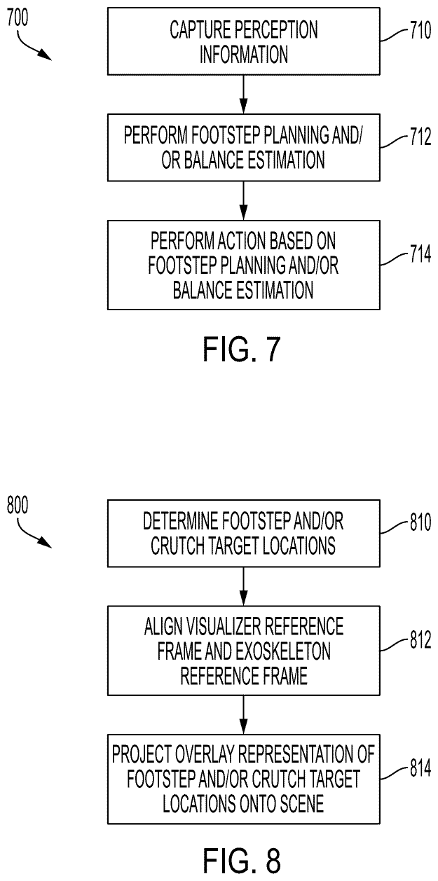

FIG. 7 is a flowchart of a process 700 for using a perception system of an exoskeleton device to perform an action, in accordance with some embodiments of the present disclosure. Process 700 begins in act 710, where perception information is captured by one or more sensors. For example, as described above, perception information may include one or more images of the terrain located in front of the exoskeleton device as captured by a camera (e.g., a camera mounted to the pelvis of the exoskeleton device). In some embodiments, perception information may include foot force information captured by a force sensor arranged on a foot of the exoskeleton device. Process 700 then proceeds to act 712, where footstep planning and/or balance estimation is performed based, at least in part, on the images captured by the camera. For instance, information from the camera and or an associated IMU may be used to generate a terrain map of the environment, and footstep planning that takes into consideration characteristics of the exoskeleton device and aspects of the terrain map may be used to determine footstep target locations and/or crutch target locations likely to result in a balance state of the exoskeleton device and its rider that is sufficient to prevent the exoskeleton device from falling over. As described above, to facilitate stable placement of the feet of the exoskeleton device on the perceived terrain, footstep planning may involve restricting footstep targets to location on the terrain where the entire foot of the exoskeleton device is in contact with the terrain surface. Additionally, foot force information sensed from the foot force sensor may be used, at least in part, to determine the balance state of the exoskeleton device and its rider.

Process 700 then proceeds to act 714, where an action is performed based on the footstep planning and/or the balance estimation performed in act 712. As described above, in some embodiments, the action performed may include setting one or more gait parameters of the exoskeleton device used to execute a next step. Such information may be provided to a controller of the exoskeleton device, wherein the controller is configured to control one or more actuators of the exoskeleton device to execute the next step. In some embodiments, the action performed in act 714 includes providing feedback to the user. For example, the feedback may enable the user to understand a current balance state of the exoskeleton device and/or how to improve the balance state to enable execution of a next step using the exoskeleton device. Additionally or alternatively the feedback may provide the user with a better understanding of future movements of the exoskeleton device based on the footstep planning. For instance, information associated with the footstep planning process (e.g., footstep target locations and/or crutch target locations) may be provided to a visualizer (e.g., an AR system) that enables the user to visualize where the exoskeleton device should step next to provide a good balance state. The visualization may also include crutch placement locations that show where the user should place the crutches to maintain a good balance state.

FIG. 8 is a flowchart of a process 800 for generating a visualization of the footstep target locations and/or crutch target locations, in accordance with some embodiments of the present disclosure. Process 800 begins in act 810, where footstep target locations and/or crutch target locations are determined. For example, a footstep planning process based, at least in part on characteristics of the exoskeleton device and a terrain map generated based on captured images may output one or more footstep target locations and/or crutch target locations.

To enable the determined footstep target locations and/or crutch target locations to be visualized in the proper locations in the environment, process 800 proceeds to act 812, where the reference frame of the visualizer worn by the user and the reference frame of the exoskeleton device are aligned. The reference frame of the visualizer and the exoskeleton device may be aligned in any suitable way. In the example shown in FIG. 5A, a plurality of fiducials are mounted to the exoskeleton device, and the fiducials may be used to align the two reference frames. In other embodiments, an example of which is shown in FIG. 5B, the use of fiducials to facilitate alignment of the visualizer and exoskeleton reference frames may not be necessary. Rather, markers on the exoskeleton device itself (e.g., located on the pelvis of the exoskeleton device) may be sensed by the visualizer and may be used to align the reference frames. After aligning the visualizer and exoskeleton reference frames, process 800 proceeds to act 814, where an overlay representation of the footstep target locations and/or the crutch target locations is projected onto the scene using the visualizer, examples of which are shown in FIGS. 5A and 5B.

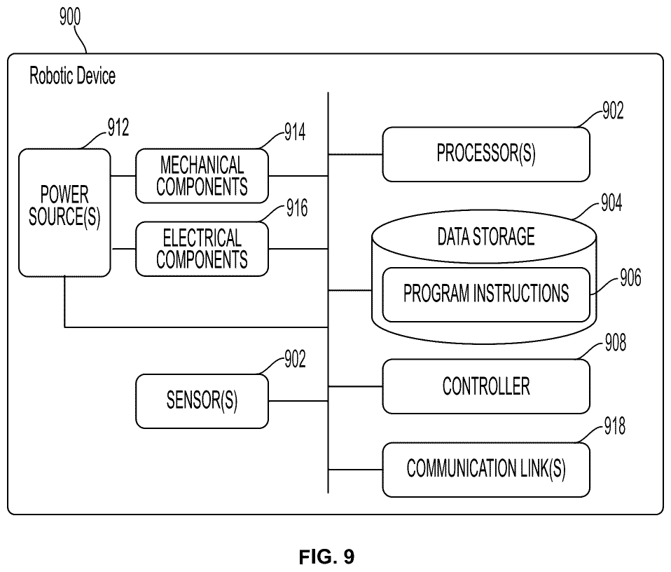

FIG. 9 illustrates an example configuration of a robotic device 900 (e.g., a lower body powered exoskeleton device as described herein), according to an illustrative embodiment of the invention. An example implementation involves a robotic device configured with at least one robotic limb, one or more sensors, and a processing system. The robotic limb may be an articulated robotic appendage including a number of members connected by joints. The robotic limb may also include a number of actuators (e.g., 2-5 actuators) coupled to the members of the limb that facilitate movement of the robotic limb through a range of motion limited by the joints connecting the members. The sensors may be configured to measure properties of the robotic device, such as angles of the joints, pressures within the actuators, joint torques, and/or positions, velocities, and/or accelerations of members of the robotic limb(s) at a given point in time. The sensors may also be configured to measure an orientation (e.g., a body orientation measurement) of the body of the robotic device (which may also be referred to herein as the “base” of the robotic device). Other example properties include the masses of various components of the robotic device, among other properties. The processing system of the robotic device may determine the angles of the joints of the robotic limb, either directly from angle sensor information or indirectly from other sensor information from which the joint angles can be calculated. The processing system may then estimate an orientation of the robotic device based on the sensed orientation of the base of the robotic device and the joint angles.

An orientation may herein refer to an angular position of an object. In some instances, an orientation may refer to an amount of rotation (e.g., in degrees or radians) about three axes. In some cases, an orientation of a robotic device may refer to the orientation of the robotic device with respect to a particular reference frame, such as the ground or a surface on which it stands. An orientation may describe the angular position using Euler angles, Tait-Bryan angles (also known as yaw, pitch, and roll angles), and/or Quaternions. In some instances, such as on a computer-readable medium, the orientation may be represented by an orientation matrix and/or an orientation quaternion, among other representations.

In some scenarios, measurements from sensors on the base of the robotic device may indicate that the robotic device is oriented in such a way and/or has a linear and/or angular velocity that requires control of one or more of the articulated appendages in order to maintain balance of the robotic device. In these scenarios, however, it may be the case that the limbs of the robotic device are oriented and/or moving such that balance control is not required. For example, the body of the robotic device may be tilted to the left, and sensors measuring the body's orientation may thus indicate a need to move limbs to balance the robotic device; however, one or more limbs of the robotic device may be extended to the right, causing the robotic device to be balanced despite the sensors on the base of the robotic device indicating otherwise. The limbs of a robotic device may apply a torque on the body of the robotic device and may also affect the robotic device's center of mass. Thus, orientation and angular velocity measurements of one portion of the robotic device may be an inaccurate representation of the orientation and angular velocity of the combination of the robotic device's body and limbs (which may be referred to herein as the “aggregate” orientation and angular velocity).

In some implementations, the processing system may be configured to estimate the aggregate orientation and/or angular velocity of the entire robotic device based on the sensed orientation of the base of the robotic device and the measured joint angles. The processing system has stored thereon a relationship between the joint angles of the robotic device and the extent to which the joint angles of the robotic device affect the orientation and/or angular velocity of the base of the robotic device. The relationship between the joint angles of the robotic device and the motion of the base of the robotic device may be determined based on the kinematics and mass properties of the limbs of the robotic devices. In other words, the relationship may specify the effects that the joint angles have on the aggregate orientation and/or angular velocity of the robotic device. Additionally, the processing system may be configured to determine components of the orientation and/or angular velocity of the robotic device caused by internal motion and components of the orientation and/or angular velocity of the robotic device caused by external motion. Further, the processing system may differentiate components of the aggregate orientation in order to determine the robotic device's aggregate yaw rate, pitch rate, and roll rate (which may be collectively referred to as the “aggregate angular velocity”).

In some implementations, the robotic device may also include a control system that is configured to control the robotic device on the basis of a simplified model of the robotic device. The control system may be configured to receive the estimated aggregate orientation and/or angular velocity of the robotic device, and subsequently control one or more jointed limbs of the robotic device to behave in a certain manner (e.g., maintain the balance of the robotic device).

In some implementations, the robotic device may include force sensors that measure or estimate the external forces (e.g., the force applied by a limb of the robotic device against the ground) along with kinematic sensors to measure the orientation of the limbs of the robotic device. The processing system may be configured to determine the robotic device's angular momentum based on information measured by the sensors. The control system may be configured with a feedback-based state observer that receives the measured angular momentum and the aggregate angular velocity, and provides a reduced-noise estimate of the angular momentum of the robotic device. The state observer may also receive measurements and/or estimates of torques or forces acting on the robotic device and use them, among other information, as a basis to determine the reduced-noise estimate of the angular momentum of the robotic device.

In some implementations, multiple relationships between the joint angles and their effect on the orientation and/or angular velocity of the base of the robotic device may be stored on the processing system. The processing system may select a particular relationship with which to determine the aggregate orientation and/or angular velocity based on the joint angles. For example, one relationship may be associated with a particular joint being between 0 and 90 degrees, and another relationship may be associated with the particular joint being between 91 and 180 degrees. The selected relationship may more accurately estimate the aggregate orientation of the robotic device than the other relationships.

In some implementations, the processing system may have stored thereon more than one relationship between the joint angles of the robotic device and the extent to which the joint angles of the robotic device affect the orientation and/or angular velocity of the base of the robotic device. Each relationship may correspond to one or more ranges of joint angle values (e.g., operating ranges). In some implementations, the robotic device may operate in one or more modes. A mode of operation may correspond to one or more of the joint angles being within a corresponding set of operating ranges. In these implementations, each mode of operation may correspond to a certain relationship.

The angular velocity of the robotic device may have multiple components describing the robotic device's orientation (e.g., rotational angles) along multiple planes. From the perspective of the robotic device, a rotational angle of the robotic device turned to the left or the right may be referred to herein as “yaw.” A rotational angle of the robotic device upwards or downwards may be referred to herein as “pitch.” A rotational angle of the robotic device tilted to the left or the right may be referred to herein as “roll.” Additionally, the rate of change of the yaw, pitch, and roll may be referred to herein as the “yaw rate,” the “pitch rate,” and the “roll rate,” respectively.

As shown in FIG. 9, the robotic device 900 includes processor(s) 902, data storage 904, program instructions 906, controller 908, sensor(s) 910, power source(s) 912, mechanical components 914, and electrical components 916. The robotic device 900 is shown for illustration purposes and may include more or fewer components without departing from the scope of the disclosure herein. The various components of robotic device 900 may be connected in any manner, including via electronic communication means, e.g., wired or wireless connections. Further, in some examples, components of the robotic device 900 may be positioned on multiple distinct physical entities rather on a single physical entity. Other example illustrations of robotic device 900 may exist as well.

Processor(s) 902 may operate as one or more general-purpose processor or special purpose processors (e.g., digital signal processors, application specific integrated circuits, etc.). The processor(s) 902 can be configured to execute computer-readable program instructions 906 that are stored in the data storage 904 and are executable to provide the operations of the robotic device 900 described herein. For instance, the program instructions 906 may be executable to provide operations of controller 908, where the controller 908 may be configured to cause activation and/or deactivation of the mechanical components 914 and the electrical components 916. The processor(s) 902 may operate and enable the robotic device 900 to perform various functions, including the functions described herein.

The data storage 904 may exist as various types of storage media, such as a memory. For example, the data storage 904 may include or take the form of one or more computer-readable storage media that can be read or accessed by processor(s) 902. The one or more computer-readable storage media can include volatile and/or non-volatile storage components, such as optical, magnetic, organic or other memory or disc storage, which can be integrated in whole or in part with processor(s) 902. In some implementations, the data storage 904 can be implemented using a single physical device (e.g., one optical, magnetic, organic or other memory or disc storage unit), while in other implementations, the data storage 904 can be implemented using two or more physical devices, which may communicate electronically (e.g., via wired or wireless communication). Further, in addition to the computer-readable program instructions 906, the data storage 904 may include additional data such as diagnostic data, among other possibilities.

The robotic device 900 may include at least one controller 908, which may interface with the robotic device 900. The controller 908 may serve as a link between portions of the robotic device 900, such as a link between mechanical components 914 and/or electrical components 916. In some instances, the controller 908 may serve as an interface between the robotic device 900 and another computing device. Furthermore, the controller 908 may serve as an interface between the robotic device 900 and a user(s). The controller 908 may include various components for communicating with the robotic device 900, including one or more joysticks or buttons, among other features. The controller 908 may perform other operations for the robotic device 900 as well. Other examples of controllers may exist as well.

Additionally, the robotic device 900 includes one or more sensor(s) 910 such as force sensors, proximity sensors, motion sensors, load sensors, position sensors, touch sensors, depth sensors, ultrasonic range sensors, and/or infrared sensors, among other possibilities. The sensor(s) 910 may provide sensor data to the processor(s) 902 to allow for appropriate interaction of the robotic device 900 with the environment as well as monitoring of operation of the systems of the robotic device 900. The sensor data may be used in evaluation of various factors for activation and deactivation of mechanical components 914 and electrical components 916 by controller 908 and/or a computing system of the robotic device 900.

The sensor(s) 910 may provide information indicative of the environment of the robotic device for the controller 908 and/or computing system to use to determine operations for the robotic device 900. Further, the robotic device 900 may include other sensor(s) 910 configured to receive information indicative of the state of the robotic device 900, including sensor(s) 910 that may monitor the state of the various components of the robotic device 900. The sensor(s) 910 may measure activity of systems of the robotic device 900 and receive information based on the operation of the various features of the robotic device 900, such the operation of extendable legs, arms, or other mechanical and/or electrical features of the robotic device 900. The sensor data provided by the sensors may enable the computing system of the robotic device 900 to determine errors in operation as well as monitor overall functioning of components of the robotic device 900.

For example, the computing system may use sensor data to determine the stability of the robotic device 900 during operations as well as measurements related to power levels, communication activities, components that require repair, among other information. As an example configuration, the robotic device 900 may include gyroscope(s), accelerometer(s), and/or other possible sensors to provide sensor data relating to the state of operation of the robotic device. Further, sensor(s) 910 may also monitor the current state of a function that the robotic device 900 may currently be operating. Additionally, the sensor(s) 910 may measure a distance between a given robotic limb of a robotic device and a center of mass of the robotic device. Other example uses for the sensor(s) 910 may exist as well.

Additionally, the robotic device 900 may also include one or more power source(s) 912 configured to supply power to various components of the robotic device 900. Among possible power systems, the robotic device 900 may include a hydraulic system, electrical system, batteries, and/or other types of power systems. As an example illustration, the robotic device 900 may include one or more batteries configured to provide power to components via a wired and/or wireless connection. Within examples, components of the mechanical components 914 and electrical components 916 may each connect to a different power source or may be powered by the same power source. Components of the robotic device 900 may connect to multiple power sources as well.

Within example configurations, any type of power source may be used to power the robotic device 900, such as a gasoline and/or electric engine. Further, the power source(s) 912 may charge using various types of charging, such as wired connections to an outside power source, wireless charging, combustion, or other examples. Other configurations may also be possible. Additionally, the robotic device 900 may include a hydraulic system configured to provide power to the mechanical components 914 using fluid power. Components of the robotic device 900 may operate based on hydraulic fluid being transmitted throughout the hydraulic system to various hydraulic motors and hydraulic cylinders, for example. The hydraulic system of the robotic device 900 may transfer a large amount of power through small tubes, flexible hoses, or other links between components of the robotic device 900. Other power sources may be included within the robotic device 900.

Mechanical components 914 can represent hardware of the robotic device 900 that may enable the robotic device 900 to operate and perform physical functions. As a few examples, the robotic device 900 may include actuator(s), extendable leg(s), arm(s), wheel(s), one or multiple structured bodies for housing the computing system or other components, and/or other mechanical components. The mechanical components 914 may depend on the design of the robotic device 900 and may also be based on the functions and/or tasks the robotic device 900 may be configured to perform. As such, depending on the operation and functions of the robotic device 900, different mechanical components 914 may be available for the robotic device 900 to utilize. In some examples, the robotic device 900 may be configured to add and/or remove mechanical components 914, which may involve assistance from a user and/or other robotic device.

The electrical components 916 may include various components capable of processing, transferring, providing electrical charge or electric signals, for example. Among possible examples, the electrical components 916 may include electrical wires, circuitry, and/or wireless communication transmitters and receivers to enable operations of the robotic device 900. The electrical components 916 may interwork with the mechanical components 914 to enable the robotic device 900 to perform various operations. The electrical components 916 may be configured to provide power from the power source(s) 912 to the various mechanical components 914, for example. Further, the robotic device 900 may include electric motors. Other examples of electrical components 916 may exist as well.

In some implementations, the robotic device 900 may also include communication link(s) 918 configured to send and/or receive information. The communication link(s) 918 may transmit data indicating the state of the various components of the robotic device 900. For example, information read in by sensor(s) 910 may be transmitted via the communication link(s) 918 to a separate device. Other diagnostic information indicating the integrity or health of the power source(s) 912, mechanical components 914, electrical components 916, processor(s) 902, data storage 904, and/or controller 908 may be transmitted via the communication link(s) 918 to an external communication device.

In some implementations, the robotic device 900 may receive information at the communication link(s) 918 that is processed by the processor(s) 902. The received information may indicate data that is accessible by the processor(s) 902 during execution of the program instructions 906, for example. Further, the received information may change aspects of the controller 908 that may affect the behavior of the mechanical components 914 or the electrical components 916. In some cases, the received information indicates a query requesting a particular piece of information (e.g., the operational state of one or more of the components of the robotic device 900), and the processor(s) 902 may subsequently transmit that particular piece of information back out the communication link(s) 918.

In some cases, the communication link(s) 918 include a wired connection. The robotic device 900 may include one or more ports to interface the communication link(s) 918 to an external device. The communication link(s) 918 may include, in addition to or alternatively to the wired connection, a wireless connection. Some example wireless connections may utilize a cellular connection, such as CDMA, EVDO, GSM/GPRS, or 4G telecommunication, such as WiMAX or LTE. Alternatively or in addition, the wireless connection may utilize a Wi-Fi connection to transmit data to a wireless local area network (WLAN). In some implementations, the wireless connection may also communicate over an infrared link, radio, Bluetooth, or a near-field communication (NFC) device.

A number of implementations have been described. Nevertheless, it will be understood that various modifications may be made without departing from the spirit and scope of the disclosure.

1. A perception system for a lower body powered exoskeleton device, the perception system comprising:
a camera configured to capture one or more images of terrain in proximity to the exoskeleton device; and
at least one processor programmed to:
perform footstep planning for the exoskeleton device based, at least in part, on the captured one or more images of terrain; and
issue an instruction to perform a first action based, at least in part, on the footstep planning.

2. The perception system of claim 1, wherein issuing an instruction to perform a first action comprises issuing an instruction to provide feedback to a user of the exoskeleton device based, at least in part on the footstep planning.

3. The perception system of claim 2, wherein issuing an instruction comprises sending information to a visualization system configured to provide the feedback to the user.

4. The perception system of claim 3, wherein
performing footstep planning for the exoskeleton device comprises determining a location of one or more footstep targets, and
sending information to the visualization system comprises sending information associated with the location of the one or more footstep targets.

5. The perception system of claim 3, wherein
performing footstep planning for the exoskeleton device comprises determining a location of one or more crutch targets to place a crutch configured to be used with the exoskeleton device, and
sending information to the visualization system comprises sending information associated with the location of the one or more crutch targets.

6. The perception system of claim 2, wherein issuing an instruction to provide feedback comprises one or more of:
issuing an instruction to an audio system to output audio including the feedback,
issuing an instruction to a haptic system to output haptic feedback to the user, or
issuing an instruction to provide at least two of visual, audio, and haptic feedback to the user.

7. The perception system of claim 1, wherein issuing an instruction to perform a first action comprises issuing an instruction to a controller of the exoskeleton device to set one or more gait parameters of the exoskeleton device based, at least in part on the footstep planning,
wherein the one or more gait parameters of the exoskeleton are selected from the group consisting of step length, step height, and step timing.

8. The perception system of claim 1, wherein performing footstep planning comprises determining at least one footstep target location for the exoskeleton device in which a foot of exoskeleton device has full contact with a surface of the terrain.

9. The perception system of claim 1, wherein
the terrain includes a step, and
performing footstep planning comprises determining at least one footstep target location for the exoskeleton device on the step.

10. The perception system of claim 1, wherein the at least one processor is further programmed to:
perform balance estimation associated with the exoskeleton device; and
issue an instruction to perform a second action based, at least in part, on the balance estimation.

11. The perception system of claim 10, wherein issuing an instruction to perform the second action comprises issuing an instruction to provide feedback to a user of the exoskeleton device based, at least in part, on the balance estimation.

12. The perception system of claim 11, wherein issuing an instruction to provide feedback comprises sending information to a visualization system configured to display the feedback to the user.

13. The perception system of claim 12, wherein
performing balance estimation associated with the exoskeleton device comprises determining a balance state associated with the exoskeleton device, and
sending information to the visualization system comprises sending information associated with the balance state.

14. The perception system of claim 13, wherein
determining a balance state associated with the exoskeleton device comprises determining a numerical value for the balance state associated with the exoskeleton device, and
the information associated with the balance state comprises information associated with the numerical value.

15. The perception system of claim 14, wherein the information associated with the numerical value comprises a balance meter that indicates to the user whether the balance state is sufficient to perform a next leg swing of the exoskeleton device.

16. The perception system of claim 15, wherein the information associated with the balance state further comprises information of how to improve the balance state when it is determined that the balance state is not sufficient to perform a next leg swing of the exoskeleton device.

17. The perception system of claim 13, wherein determining a balance state of the exoskeleton device is performed, based at least in part, on force information received from at least one sensor.

18. The perception system of claim 17, wherein
the at least one sensor includes a foot force sensor located on a foot portion of the exoskeleton device, and
the force information comprises force information received from the foot force sensor.

19. The perception system of claim 17, wherein
the at least one sensor includes a force sensor arranged on a crutch configured to be used with the exoskeleton device, and
the force information comprises force information received from the force sensor arranged on the crutch.

20. The perception system of claim 2, further comprising an inertial measurement unit (IMU),
wherein issuing an instruction to provide feedback to the user of the exoskeleton device comprises issuing an instruction to provide feedback based, at least in part on an output of the IMU.

21. A method of providing assistive feedback to a user of a lower body powered exoskeleton device, the method comprising:
receiving one or more images of terrain in front of the exoskeleton device;
performing, by at least one processor, footstep planning for the exoskeleton device, the footstep planning being performed based, at least in part, on the one or more images of terrain; and
providing assistive feedback to the user of the exoskeleton device based, at least in part, on the footstep planning.

22. A system, comprising:
a lower body powered exoskeleton device configured to be worn by a user;
a perception system configured to capture one or more images of terrain in proximity to the exoskeleton device;
an augmented reality (AR) system configured to be worn by the user; and
at least one processor programmed to:
perform footstep planning for the exoskeleton device based, at least in part, on the one or more images of terrain; and
send first information based on a result of the footstep planning to the AR system for presentation by the AR system to the user.

23. The system of claim 22, further comprising:
a foot force sensor configured to sense foot force information,
wherein the at least one processor is further programmed to perform balance estimation based, at least in part, on the foot force information.

24. The system of claim 23, wherein the at least one processor is further programmed to send second information based on a result of the balance estimation to the AR system for presentation by the AR system to the user.
